(19) United States
(12) Patent Application Publication
(10) Pub. No.: US 2005/0256419 A1
(43) Pub. Date: Nov. 17, 2005

(54) Cardiac coupled resipiration

(75) Inventors: Daniel E. Roach, Calgary (CA); Henry J. Duff, Calgary (CA); Robert S. Sheldon, Calgary (CA)

Correspondence Address: THOMPSON LAMBERT;SUITE 703D, CRYSTAL PARK TWO, 2121 CRYSTAL DRIVE, ARLINGTON, VA, 22202, US

(73) Assignee: University Technologies International Inc., Calgary, AL (US)

(21) Appl. No.: 10/846,576

(22) Filed: May 17, 2004

(51) Int. Cl. A61B 5/04

(52) U.S. Cl. 600/520; 600/513

(57) ABSTRACT

A series of feed-forward applications are designed to synchronize heart/respiration/muscle and brain to produce a better quality of life endpoint. The applications will be used to treat anxiety, sleep disorders, arrhythmias and enhanced performance during athletic training.

### BACKGROUND OF THE INVENTION

Abnormalities of autonomic nervous system function underlie a number of medical conditions that contribute to substantial morbidity including anxiety, sleep disorders, and arrhythmias. Individuals with anxiety, panic attacks and sleep disorders experience impaired role functioning, spend more days in bed due to illness, have more work days, lost, have increased impairment at work, and high use of health services. These problems are common, likely occurring in 2-5% of the general population in North America or Europe. The disability of anxiety is just as great as that caused by other common medical conditions, such as hypertension, diabetes and arthritis. Pharmacologic treatment of these conditions is fraught with side effects, non-compliance and inefficacy.

Improvements in human physiological performance due to coupling of human physiological activities with other human physiological activities is an area of much current study. For example, Kullock, U.S. Pat. No. 6,644,976 issued Nov. 11, 2003, discusses time correlation of movements and physiological components. Kullock requires a synergic programs module that directs physiological movement according to dual correlation factors. This invention is directed towards an improvement in cardiac coupling with physiological activity, for entraining heart, respiration, muscles and brain, that does not require complicated equipment.

### SUMMARY OF THE INVENTION

There is therefore provided, in accordance with an aspect of the invention, a method of controlling a cardiac cycle of a human being by detecting the cardiac cycle and intentionally coordinating a physiological activity with the cardiac cycle. The physiological activity, for example the respiratory cycle, is preferably matched to a point in the cardiac cycle such as the QRS of the cardiac cycle. The invention therefore provides a phase-locked loop methodology so that an individual can customize their respiratory cycle (wavelength and inhalation/expiration symmetry) to produce an effect on the cardiac cycle.

Operation of the method may be enhanced using a heart monitor to measure the cardiac cycle and a device to measure the physiological activity, such as a respiratory cycle monitor. Coordination of the cycles may be enhanced by generating a signal coordinated with the cardiac cycle, or by monitoring synchronicity between the cardiac cycle and the physiological activity. Other physiological activity that may be coupled are a hand movement, whole body movement or response to an auditory or visual signal.

Cardiac coupled respiration may be useful to lower blood pressure, reduce nervousness as indicated by a quiverometer, lengthen the RR-interval (lower heart rate), allow relaxation in preparation for sleep and increased sport performance. Subjects may be provided with instruction sheets for carrying out the method steps of the invention, or may be instructed in a clinic setting.

These and other aspects of the invention are set out in the claims, which are incorporated here by reference.

### BRIEF DESCRIPTION OF THE FIGURES

Preferred embodiments of the invention will now be described with reference to the figures, in which like reference characters denote like elements, by way of example, and in which:

FIG. 1 is a flow diagram showing the basic process steps and including a representation of the cardiac cycle of a human being;

FIG. 2 is a graph showing the effect of cardiac coupled respiration;

FIG. 3A is a graph showing synchronicity between a cardiac cycle and respiration of a human being carrying out cardiac coupled respiration;

FIG. 3B illustrates an embodiment of the invention with feedback;

FIG. 4 shows two graphs indicating the effect of cardiac coupled respiration on blood pressure and RR-interval of a human being;

FIG. 5 shows effect on cardiac coupled respiration of addition of a muscle-heart reflex; and

FIG. 6 illustrates equipment useful for carrying out the invention.

### DETAILED DESCRIPTION OF PREFERRED EMBODIMENTS

In the claims, the word “comprising” is used in its inclusive sense and does not exclude other elements being present. The indefinite article “a” before a claim feature does not exclude more than one of the feature being present.

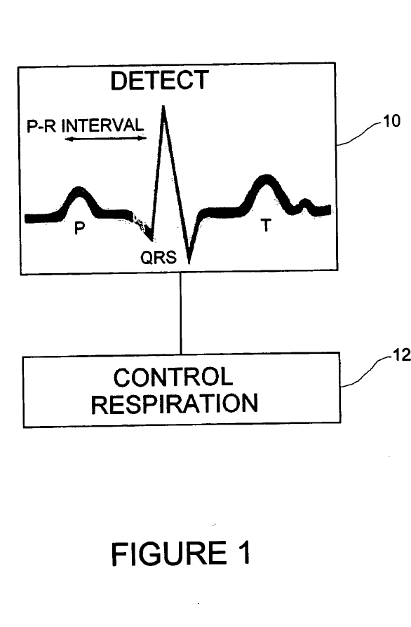

Referring to FIG. 1, in a first method step 10 of the invention, the cardiac cycle is detected. As shown in FIG. 1, the electrical cardiac cycle of a human being follows a well established pathway from the P through the QRS to the T parts of the cycle. The P part of the cycle corresponds to depolarization of the atrium by the sinus node of the heart, and is followed after the P-R interval, typically about 0.2 seconds later, by the QRS, which corresponds to the depolarization of the ventricular. This cycle is controlled by activation of the sinus node, which causes a depolarization wave to spread through the heart and cause the heart muscles to contract and pump blood. The electrical cardiac cycle can be detected directly by a device such as an electrocardiogram. As the heart muscle contracts, the cardiac cycle may be detected by the human being as a pulsation, for example by placing pressure on an artery using a finger, or may be detected by such devices as a heart monitor. The RR interval in a cardiac cycle is the time between R parts of successive cycles.

Control of the cardiac cycle is effected by following the step 10 of detecting the cardiac cycle, as for example by the methods described in the immediately preceding paragraph, with the step 12 of intentionally coordinating a physiological activity with the cardiac cycle. An example of a physiological activity is the respiratory cycle of the human being. For example, the human being may control breathing to match a peak of inspiration or expiration with a point in the cardiac cycle. This makes the respiratory cycle in phase and synchronized with the cardiac cycle. Since the human respiration rate is much lower in frequency than the cardiac cycle, the frequency of the cardiac cycle should be a harmonic of the respiration frequency, as for example the fourth of fifth harmonic. For application of the method to other animals, other harmonics are applicable. The point in the cardiac cycle may be the QRS of the cardiac cycle for ease of detection.

Coordination of the cardiac cycle with for example breathing as a representative physiological activity may be readily carried out using an instrument such as a heart monitor to detect the cardiac signal, generating a visual or audible signal coordinated with the cardiac cycle and having the subject actively coordinate breathing with the visual or audible signal. For example, as the cardiac cycle reaches the QRS point, a signal may be given, thus enabling the subject to more readily match the point of maximum expiration or inspiration to the QRS.

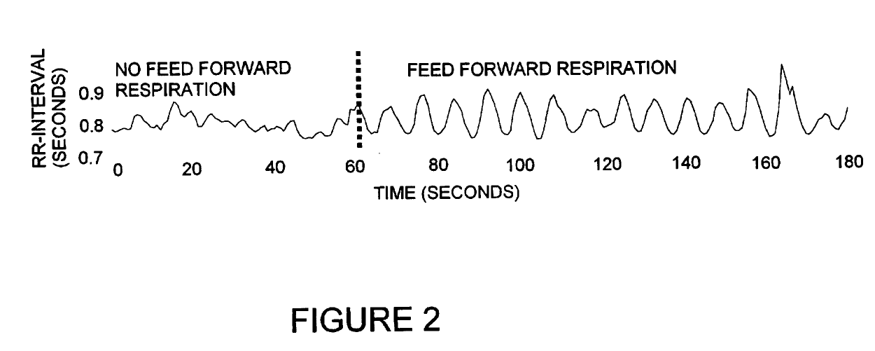

Response of the cardiac cycle to matching of breathing with the cardiac cycle is illustrated in FIG. 2. For the generation of FIG. 2, the subject rested quietly for 60 seconds, breathing spontaneously. The section of the graph indicated by “No Feed Forward Respiration” shows the irregular nature of the RR-interval sequence. After 60 seconds, the subject began feed forward respiration wherein end expiration was coupled to a QRS, and end inspiration was also coupled to a QRS. The subject repeatedly inhaled for 5 heart beats, and exhaled for 5 heart beats. As shown in FIG. 2, the RR-interval sequence quickly becomes periodic with a 0.1 Hz frequency.

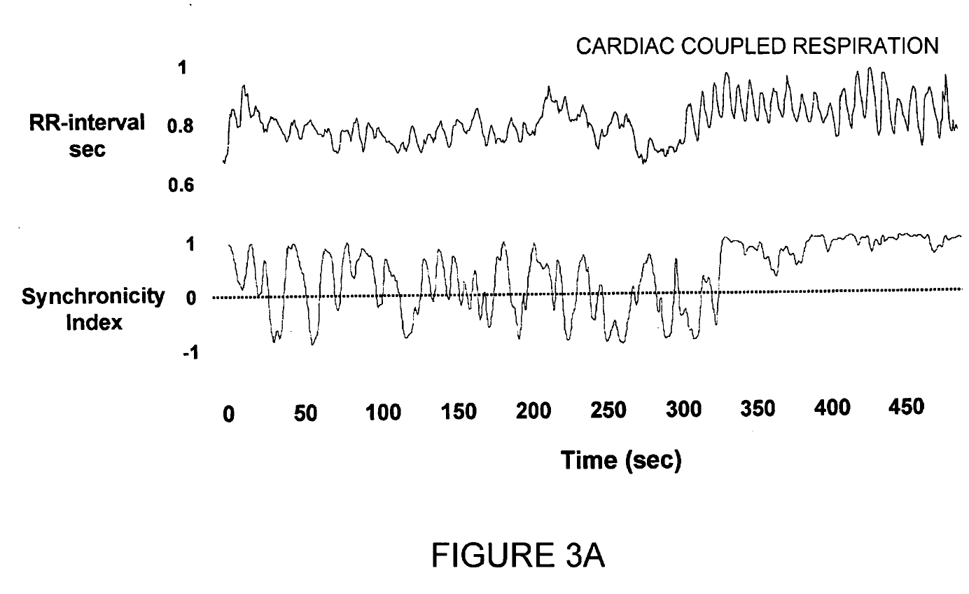

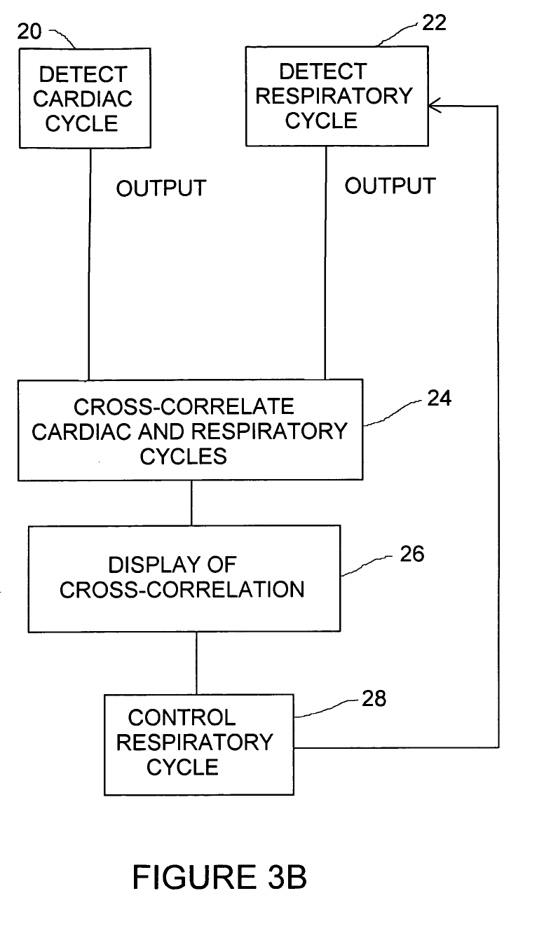

Coordination of the cardiac cycle with the physiological activity may be monitored to improve the coordination. Various methods may be used to measure the extent of coordination of the cardiac cycle with the physiological activity. For example, the respiratory cycle may be monitored using any of various devices such as a respiratory volume sensor. The respiratory volume sensor yields a time sequence of data whose value is an indication of respiratory volume. This time sequence of data may be correlated with a signal from a heart monitor to yield a synchronicity index. FIG. 3A shows a synchronicity index. The upper trace shows the RR-interval in seconds. The lower trace shows the synchronicity index found by a moving auto-correlation of the electric cardiac cycle. Synchronicity may also be found from cross-correlating respiratory volume with the cardiac cycle. For 300 seconds, the subject was at rest, with no cardiac coupled breathing. After 300 seconds, cardiac coupled respiration commenced, and the upper trace shows the evident greater periodicity of the RR-interval arising from cardiac coupled respiration. The lower trace shows the result of the cross-correlation. After 300 seconds, correlation values are near 1, indicating a high degree of coupling between the two signals. Improvement in the coupling may be obtained by varying the controlled variable, namely respiration, to achieve a high degree of synchronicity.

FIG. 3B illustrates the process steps. In step 20, the cardiac cycle is detected with for example a heart monitor. In step 22, the respiratory cycle is detected with for example a respiratory volume monitor. Output from the monitors is supplied to a processor, such as a computer, to cross-correlate the two outputs in step 24. A display, such as a visual or audible display, is used to display the cross-correlation in step 26. Various methods may be used to show the degree of cross-correlation such as the graph of FIG. 3A, or intensity, eg higher intensity meaning greater correlation, or sound, eg a louder sound indicating greater correlation. The subject may then vary the respiratory cycle, slowing or speeding up respiration, in step 28 using the displayed correlation to achieve a higher degree of correlation.

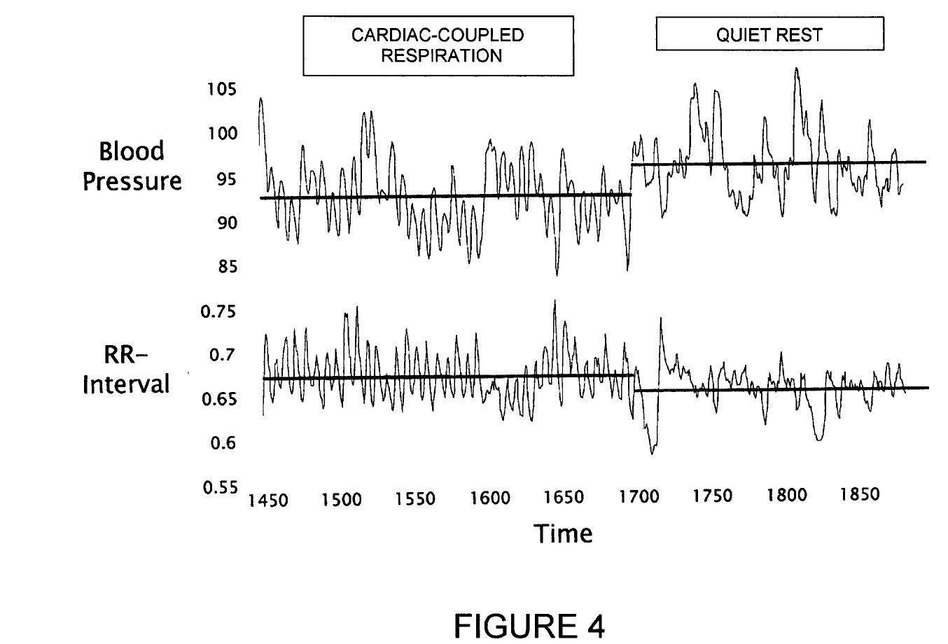

It has been found that cardiac coupled respiration, in at least some subjects, reduces blood pressure, increases the mean RR-interval and reduces nervousness as indicated by a quiverometer. The response of blood pressure, RR-interval, nervousness or other physiological parameter to the cardiac coupled respiration may be found by monitoring the respective physiological parameter with a suitable monitor, such as a blood pressure monitor, ECG, or quiverometer, respectively. A quiverometer is a device that measures involuntary muscle tremor, and may for example consist of a rod fixed to the end of a person's finger with an indicating point at the end of the rod, the location of which may be tracked by any suitable means. The monitor may be arranged to give a signal once a desired response of the physiological parameter has been achieved. FIG. 4 shows, upper trace, (A) reduction of blood pressure due to cardiac coupled respiration as compared with quiet rest and (B) increased RR-interval due to cardiac coupled respiration as compared with quiet rest. Lower blood pressure and increased RR-interval are generally considered to be beneficial for human beings, although the work underlying this patent document has not involved investigating improvements in health of subjects, other than measurement of these vital signs. Due to the increased RR-interval, reduced blood pressure and lower nervousness, cardiac coupled respiration is believed to be particularly useful when a subject is preparing for sleep, or carrying out a physical activity, such as a sport. Cardiac coupled respiration is believed to be particularly for a sport such as golf in which uniform repeated motions under low nervous activity are desirable.

It is believed that other physiological activities that cause a stimulus to the sinus node of the heart (muscle-heart reflex) will have a similar effect to respiration when coupled with the cardiac cycle. For example, physiological activities such as a hand movement, where a hand is caused to periodically grip and ungrip an object, or a whole body movement such as rocking a rocking chair, or an auditory or visual response to a repeated auditory or visual signal respectively are all believed to be capable of causing cardiac coupling.

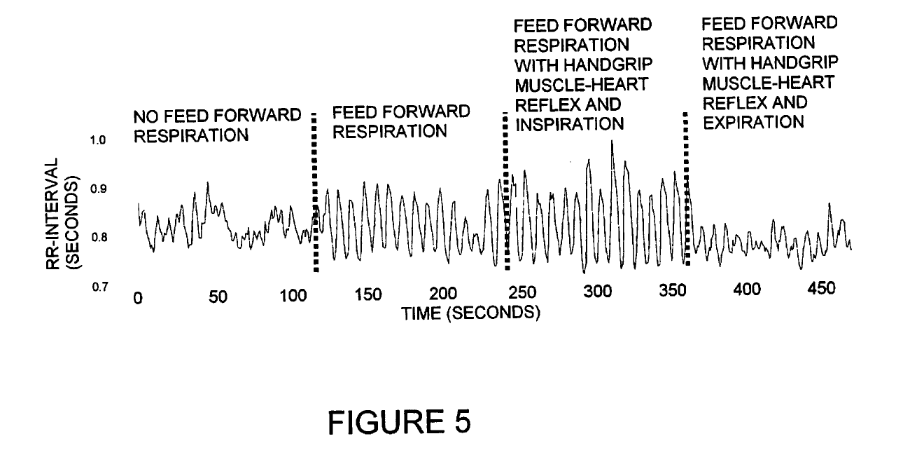

FIG. 5 illustrates the combined effects of feed forward respiration and muscle-heart reflex. For the first 60 seconds, the subject rested quietly breathing spontaneously. The RR-interval sequence is relatively irregular. After 120 seconds, the subject began feed forward respiration, where he repeatedly inhaled for 5 heart beats and exhaled for 5 heart beats. The RR-interval sequence quickly became periodic with a periodicity of ˜10 seconds, (I.e., frequency equal to ˜0.1 Hz). At 240 seconds, the subject began to squeeze a hand-grip for for the first 2 heart beats of the inhalation phase of respiration. The magnitude of the 0.1 Hz oscillations was increased. At 360 seconds, the subject began to squeeze a hand-grip for the first two beats of the expiration phase. The magnitude of the 0.1 oscillation was greatly reduced.

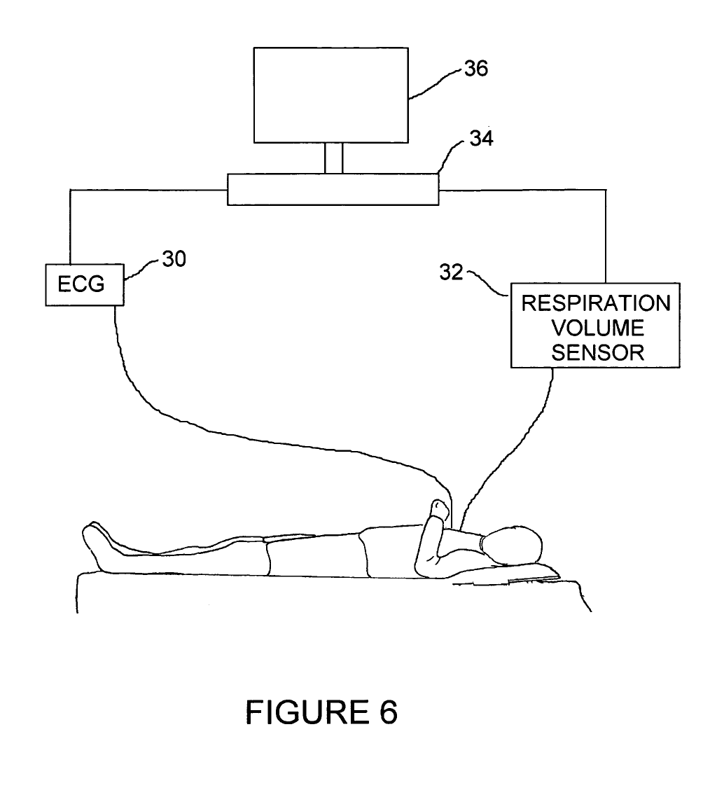

Referring to FIG. 6, various apparatus may be useful in effecting cardiac coupled physiological activity including a cardiac cycle monitor, such as an ECG apparatus 30, having a representation of the cardiac cycle as output. Also useful is a sensor of the physiological activity, having a representation of the physiological activity as output. A physiological sensor may also use a representation of the cardiac cycle as input, if the cardiac cycle also contains information about the physiological activity. For example, the cardiac cycle contains information about the respiratory cycle, and the sensor may be used to detect this information. A respiratory volume sensor 32 is also useful for the physiological activity sensor. To detect coordination between the physiological activity and the cardiac cycle, the outputs of the cardiac cycle monitor and the sensor may be input to a general purpose computer 34 and the two signals cross-correlated to yield the synchronization index of FIG. 3. The synchronization index may be viewed in real time on a computer monitor 36 to assist the subject in improving the coupling. By arranging for the cardiac cycle monitor to output a visual or audible signal at a recurring point in the cardiac cycle, the subject may more readily control the coupling of the physiological activity with the cardiac cycle.

Instructions sheets or other media carrying human readable instructions may be provided for instructing a person to carry out the method steps of the invention. For example, the media may direct a person to listen for an audible signal from a heart monitor that for example beeps at each 5th QRS and reach a point of deepest breathing in and then deepest breathing out at alternating beeps. Feedback for the subject may also be provided by directing the subject to monitor blood pressure and maintain a log to indicate whether there is any blood pressure reduction from following the process steps.

The control of the physiological activity that is being coordinated with the cardiac cycle is generally carried out intentionally by the subject, where the subject adverts to the cardiac cycle or a representation of the cardiac cycle, and deliberately controls the physiological activity. In the case of breathing, it is believed that coordination with the cardiac cycle may also be obtained by enforced regulation of the breathing cycle.

Immaterial modifications may be made to the embodiments of the invention described here without departing from the invention.

1. A method of controlling a cardiac cycle of a human being, the method comprising the steps of:
detecting the cardiac cycle of a human being; and
the human being intentionally coordinating a physiological activity with the cardiac cycle.

2. The method of claim 1 in which the physiological activity is the respiratory cycle of the human being.

3. The method of claim 2 in which coordinating the physiological cycle with the cardiac cycle comprises matching a peak of inspiration or expiration with a point in the cardiac cycle.

4. The method of claim 3 in which the point in the cardiac cycle is the QRS of the cardiac cycle.

5. The method of claim 1 in which detecting the cardiac cycle comprises measuring the cardiac cycle with an instrument and generating a signal coordinated with the cardiac cycle.

6. The method of claim 5 in which the signal is an audible or visible signal.

7. The method of claim 1 further comprising the step of finding an indication of the extent of coordination of the cardiac cycle with the physiological activity.

8. The method of claim 7 further comprising the step of using the indication of the extent of coordination to improve the coordination.

9. The method of claim 1 further comprising the step of monitoring a physiological parameter that is responsive to the coordination of the cardiac cycle with the physiological parameter.

10. The method of claim 9 further comprising the step of producing a signal indicative of a desired response of the physiological parameter.

11. The method of claim 1 in which the physiological activity is a hand movement.

12. The method of claim 1 in which the physiological activity is a whole body movement.

13. The method of claim 1 in which the physiological activity is a response to an auditory or visual signal.

14. The method of claim 1 carried out while the human being is preparing for sleep.

15. The method of claim 1 carried out while the human being is carrying out a physical activity.

16. Apparatus for controlling a cardiac cycle of a human being, the apparatus comprising:
a cardiac cycle monitor having a representation of the cardiac cycle as output;
a sensor of a physiological activity, having a representation of the physiological activity as output; and
a detector of coordination between the physiological activity and the cardiac cycle, the detector of coordination having the representation of the cardiac cycle and a representation of the physiological activity as input.

17. The apparatus of claim 16 in which the sensor is a respiratory volume sensor.

18. The apparatus of claim 17 in which the cardiac cycle monitor outputs a visual or audible signal at a recurring point in the cardiac cycle.

19. The apparatus of claim 18 in which the detector computes a cross-correlation between the representation of the cardiac cycle and the representation of the physiological activity.

20. A method of controlling a cardiac cycle of a human being, the method comprising the steps of:
detecting the cardiac cycle; and
coordinating respiration with the cardiac cycle.

21. The method of claim 20 in which coordinating respiration with the cardiac cycle comprises matching a peak of inspiration or expiration with a point in the cardiac cycle.

22. The method of claim 21 in which the point in the cardiac cycle is the QRS of the cardiac cycle.

23. The method of claim 20 in which detecting the cardiac cycle comprises measuring the cardiac cycle with an instrument and generating a signal coordinated with the cardiac cycle.

24. The method of claim 24 further comprising the step of finding an indication of the extent of coordination of the cardiac cycle with respiration.

25. The method of claim 24 further comprising the step of using the indication of the extent of coordination to improve the coordination.

26. The method of claim 20 further comprising the step of monitoring a physiological parameter that is responsive to the coordination of the cardiac cycle with the physiological parameter.

27. The method of claim 26 further comprising the step of producing a signal indicative of a desired response of the physiological parameter.

28. The method of claim 20 in which coordinating respiration is carried out intentionally by a human being.

29. Media carrying human readable instructions for carrying out the method of claim 1.

30. Media carrying human readable instructions for carrying out the method of claim 20.
